(19) United States
(12) Patent Application Publication
(10) Pub. No.: US 2025/0170180 A1
(43) Pub. Date: May 29, 2025

(54) TREATMENT OF CANCERS WITH A REGIMEN OF TARGETED RADIONUCLIDE THERAPY AND DUAL CAR T CELL THERAPY

(71) Applicant: Wisconsin Alumni Research Foundation, Madison, WI (US)

(72) Inventors: Zachary Morris, Madison, WI (US); Christian Capitini, Madison, WI (US); Quaovi Sodji, Madison, WI (US)

(73) Assignee: Wisconsin Alumni Research Foundation, Madison, WI (US)

(21) Appl. No.: 18/955,483

(22) Filed: Nov. 21, 2024

Related U.S. Application Data
(60) Provisional application No. 63/603,738, filed on Nov. 29, 2023.

(51) Int. Cl. A61K 35/17; A61K 39/00; A61K 51/04; C07K 16/30; C07K 16/44

(52) U.S. Cl. CPC A61K 35/17; A61K 40/11; A61K 40/31; A61K 40/4258; A61K 51/0408; C07K 16/3084; C07K 16/44; A61K 2121/00; A61K 2239/13; A61K 2239/21

(57) ABSTRACT

Described herein are genetically modified bispecific immune cells such as bicistronic and bivalent immune cells. The bispecific immune cells include an antigen recognition domain that specifically binds a tumor-specific antigen and an antigen recognition domain that specifically binds a radiation-induced cell surface marker. Also described are methods of treating cancer in a subject including administering to the subject a dose of a targeted radionuclide therapy (TRT) agent, and administering to the subject the genetically modified bispecific immune cell.

### CROSS-REFERENCE TO RELATED APPLICATIONS

This application claims priority to U.S. Provisional Application 63/603,738 filed on Nov. 29, 2023, which is incorporated herein by reference in its entirety.

### STATEMENT REGARDING FEDERALLY SPONSORED RESEARCH & DEVELOPMENT

This invention was made with government support under CA014520 awarded by the National Institutes of Health. The government has certain rights in the invention.

### FIELD OF THE DISCLOSURE

The present disclosure includes methods of treating cancers with a combination of targeted radionuclide therapy and dual chimeric antigen receptor (CAR) T cell therapy.

### BACKGROUND

CAR T cell therapy represents one of the forms of adoptive T cell transfer therapy that has revolutionized cancer immunotherapy. Structurally, a CAR is composed of an extracellular domain which includes an antigen-binding domain taken from the single-chain variable fragment (scFv) of an antibody, a transmembrane domain, and an intracellular domain comprised of a T cell signaling domain (e.g., CD3-zeta) and costimulatory domains (e.g., OX40, 4-1BB or CD28). CAR T cells targeting CD19, or B cell maturation antigen (BCMA), are FDA-approved for the treatment of relapsed/refractory B cell lymphoma/leukemia and multiple myeloma respectively, following high rates of complete responses during clinical trials.

Despite success against hematological malignancies, CAR T cell therapy has not been effective against non-hematological solid tumors for numerous reasons including lack of persistence, exhaustion, lack of infiltration into the immunosuppressive tumor microenvironment (TME), and decreased antigen expression by tumor cells.

What is needed are novel treatment regimens including CAR T cell therapy for treating cancers including solid tumors.

### BRIEF SUMMARY

In an aspect, genetically modified bispecific immune cells (including, but not limited to, T cells, NK cells, and macrophages) comprise genetically modified bicistronic immune cells and genetically modified bivalent immune cells.

In an aspect, a genetically modified bicistronic immune cell comprises a first expressed chimeric antigen receptor (CAR) construct comprising a first extracellular domain linked to a first intracellular domain through a first transmembrane domain, wherein the first extracellular domain comprises an antigen recognition domain that specifically binds a tumor-specific antigen; and a second expressed CAR construct comprising a second extracellular domain linked directly or indirectly to a second intracellular domain through a second transmembrane domain, wherein the second extracellular domain comprises an antigen recognition domain that specifically binds a radiation-induced cell surface marker.

In another aspect, a genetically modified bivalent immune cell comprises a bivalent extracellular domain which comprises a first antigen recognition domain that specifically binds a tumor-specific antigen and a second antigen recognition domain that specifically binds a radiation-induced cell surface marker, linked to a single intracellular domain through a single transmembrane domain.

In a further aspect, a method of treating a cancer in a subject comprises administering to the subject a dose of a targeted radionuclide therapy (TRT) agent, and administering to the subject the genetically modified bispecific immune cell.

### BRIEF DESCRIPTION OF THE DRAWINGS

FIG. 1 shows design of an embodiment of a dual CAR T cell with a separate CAR controlling the activation of T cell. This design includes an “AND” logic, that is, the T cell is only activated after the binding of both antigen recognition domains.

FIG. 2 is a schematic representation of a CAR T cell targeting radiation-induced cell surface antigens on malignant cells.

FIG. 3 shows conjugation of an antibody/adaptor with 2,4-dinitrophenyl (DNP) to obtain a DNP labelled antibody capable of recognizing a cell surface epitope and incorporating a CAR T binding site.

FIGS. 4A and B show representations of DNP directed CAR T cell targeting radiation-induced cell surface calreticulin. 4A) Calreticulin antibody labelled with DNP is recognized by the anti-DNP CAR on the T cell and mediates the recognition of a cell surface antigen. 4B) DNP directed CAR T cell construct.

The above-described and other features will be appreciated and understood by those skilled in the art from the following detailed description, drawings, and appended claims.

### DETAILED DESCRIPTION

As explained in the Background, CAR T cell therapy has not been very effective against some cancers such as non-hematological solid tumors. With targeted radionuclide therapy (TRT), radioactive compounds are selectively delivered to malignant cells using tumor homing ligands. TRT enables the systemic delivery of radiation to all sites of disease in patients with metastatic disease and has demonstrated a survival benefit in patients with castration-resistant prostate cancer as well as to subsets of patients with neuroblastoma. Furthermore, emerging data suggests that radiation delivered by TRT elicits a favorable immune response by activating and enhancing the infiltration of endogenous T cells into the TME. TRT may help overcome some of the shortcomings of CAR T cell therapy against non-hematological solid tumors.

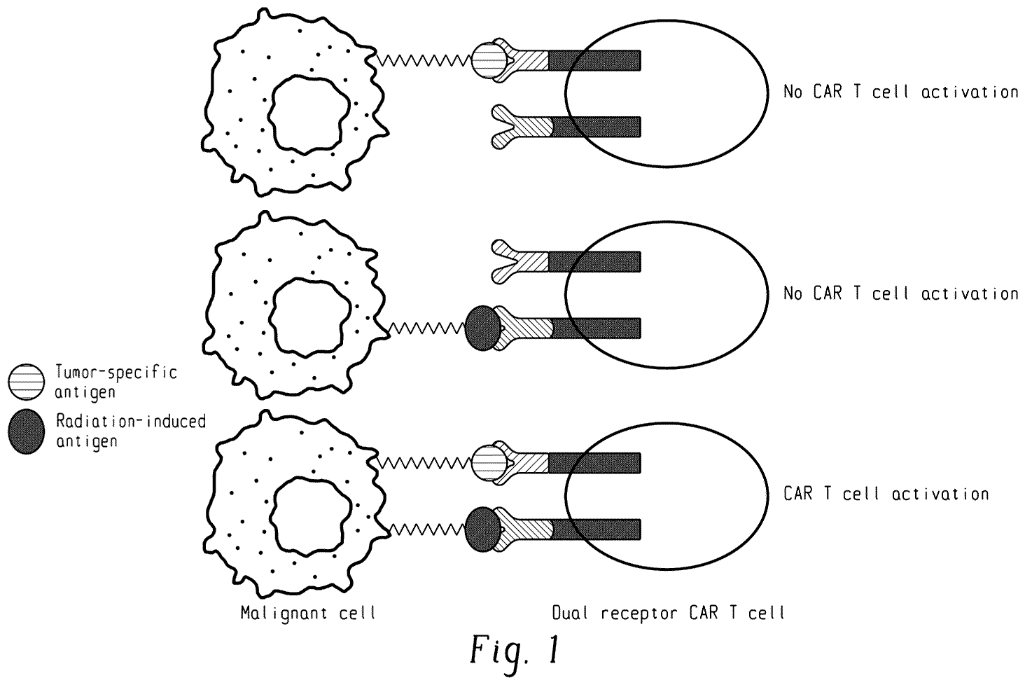

Due to the immunogenicity effects elicited by TRT, its combination with CAR T cell therapy holds therapeutic potential as it may help overcome some limitations facing CAR T cells therapy for solid tumors, including an immunosuppressive TME. Using the capacity of TRT to systemically deliver RT to the TME, a combination of immunomodulatory doses of TRT and CAR T cell therapy can be directed towards all disease sites. Also, using a dual receptor CAR design, targeting a tumor specific antigen and a radiation-induced neoantigen may enhance the safety of the combination therapy (FIG. 1).

As described herein, the tumor selectivity of TRT can be leveraged to confer greater tumor specificity, infiltration, and activation of genetically modified bivalent immune cells (e.g., dual CAR T cell therapies) that target TRT-induced markers in combination with tumor-specific surface markers. The combination of TRT and dual CAR T cells represents a novel therapeutic option for not only GD2-expressing neuroblastoma, but potentially for any patient whose tumor can be targeted by TRT and a tumor-selective surface marker.

### Genetically Modified Bivalent Immune Cells (Dual CAR T Cells)

Adoptive cell therapy with genetically modified immune cells such as CAR T cells has emerged as a novel immunotherapy approach. T cells, for example, interact specifically with the target of their T cell receptor (TCR), enabling highly specific responses which can be engineered towards novel antigens and targets by inserting a new receptor with the desired specificity into a T cell. Genetically modified immune cells include an extracellular domain comprising an antigen recognition domain linked to a first intracellular domain through a first transmembrane domain. Typically, the antigen recognition domain is a monoclonal antibody-derived single-chain variable fragment (scFv) capable of targeting an antigen such as a specific tumor-associated antigen.

Genetically modified bivalent immune cells such as dual CAR T cells can include tandem or bivalent constructs in which two distinct antigen binding sites are included in a single extracellular domain, as well as bicistronic constructs in which dual targeting is achieved using separate extracellular domains.

In an aspect, a genetically modified bicistronic immune cell comprises a first expressed chimeric antigen receptor (CAR) construct comprising a first extracellular domain linked to a first intracellular domain through a first transmembrane domain, wherein the first extracellular domain comprises an antigen recognition domain that specifically binds a tumor-specific antigen; and a second expressed CAR construct comprising a second extracellular domain linked directly or indirectly to a second intracellular domain through a second transmembrane domain, wherein the second extracellular domain comprises an antigen recognition domain that specifically binds a radiation-induced cell surface marker.

In another aspect, a genetically modified bivalent immune cell comprises a bivalent expressed CAR construct comprising a bivalent extracellular domain which comprises a first antigen recognition domain that specifically binds a tumor-specific antigen and a second antigen recognition domain that specifically binds a radiation-induced cell surface marker, linked to a single intracellular domain through a single transmembrane domain.

In some aspects, the immune cell is a T-cell, a Natural Killer (NK) cell, an innate lymphoid cell, a Cytokine Induced Killer (CIK) cell, a hematopoietic progenitor cell, a peripheral blood (PB) derived immune cell, a bone marrow derived immune cell, a macrophage, or an umbilical cord blood (UCB) derived immune cell. In some aspects, the immune cell is an embryonic or induced pluripotent stem cell (iPSC)-derived immune cell. In some aspects, wherein the immune cells are modified autologous cells isolated from a patient in need of cancer treatment, or modified cells from an allogeneic healthy donor with intent to treat a patient with cancer.

The immune cells may be isolated from subjects, particularly mammalian subjects such as human subjects and companion animals. The immune cells can be obtained from a subject of interest, such as a subject suspected of having a particular disease or condition, a subject suspected of having a predisposition to a particular disease or condition, or a subject who is undergoing therapy for a particular disease or condition. The immune cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the immune cells are enriched, isolated, and/or purified may be isolated from both living and non-living subjects, wherein the non-living subjects are organ donors. The isolated immune cells may be used directly, or they can be stored for a period of time, such as by freezing.

The population of immune cells can be obtained from a subject in need of therapy or suffering from a disease associated with reduced immune cell activity. Thus, the cells will be autologous to the subject in need of therapy. Alternatively, the population of immune cells can be obtained from a donor such as an allogenic healthy donor. The immune cell population can be harvested from PB, cord blood, bone marrow, spleen, or any other organ/tissue in which immune cells reside in said subject or donor. The immune cells can be isolated from a pool of subjects and/or donors, such as from pooled cord blood. The population of immune cells can be derived from iPSCs and/or any other stem cell known in the art. In some aspects, the iPSCs and/or stem cells used to derive the population of immune cells can be obtained from a subject in need of therapy or suffering from a disease associate with reduced immune cell activity, thus these iPSCs and/or stem cells will be autologous to the subject in need of therapy. Alternatively, the iPSCs and/or stem cells can be obtained from a healthy donor and therefore be allogeneic to the subject in need of therapy.

When the population of immune cells is obtained from a donor distinct from the subject, the donor is preferably allogeneic, provided the cells obtained are subject-compatible in that they can be introduced into the subject. Allogeneic donor cells may or may not be human leukocyte antigen (HLA)-compatible. To be rendered subject-compatible, allogeneic cells can be treated to reduce immunogenicity.

The bispecific immune cells described herein express an antigen recognition domain that binds a tumor-specific antigen and an antigen recognition domain that binds a radiation-induced cell surface marker.

The antigen-specific extracellular domain of a CAR recognizes and specifically binds an antigen, typically a surface-expressed antigen of a malignancy. An antigen-specific extracellular domain specifically binds an antigen when, for example, it binds the antigen with an affinity constant or affinity of interaction (KD) between about 0.1 pM to about 10 μM, specifically about 0.1 pM to about 1 μM, more specifically about 0.1 pM to about 100 nM. Methods for determining the affinity of interaction are known in the art. An antigen-specific extracellular domain suitable for use in a CAR may be any antigen-binding polypeptide, one or more scFv, or another antibody-based recognition domain (cAb VHH (camelid antibody variable domains) or humanized versions thereof, IgNAR VH (shark antibody variable domains) and humanized versions thereof, sdAb VH (single domain antibody variable domains) and “camelized” antibody variable domains are suitable for use. In some instances, T cell receptor (TCR) based recognition domains such as single chain TCR may be used as well as ligands for cytokine receptors.

The bispecific immune cells described herein express an antigen recognition domain that binds a tumor-specific antigen. The antigen can be expressed as a peptide or as an intact protein or portion thereof. The intact protein or a portion thereof can be native or mutagenized. Non-limiting examples of tumor-specific antigens that are CAR targets include carbonic anhydrase IX (CAIX), carcinoembryonic antigen (CEA), CD8, CD7, CD10, CD19, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), adult AChR subunits, folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MARTI, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-vIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A, ERBB, and combinations thereof.

Specific tumor-specific antigens include GD2, HER2, EGFR, mesothelin, Claudin-18.2, PSMA, B7-H3, IL-13Rα2, FAP, CA19, CD19, CD5, MUC1, or a combination thereof.

The bispecific immune cells also include an antigen recognition domain that binds a radiation-induced cell surface marker. Calreticulin, for example, is a peptide that resides in the endoplasmic reticulum (ER) under normal physiological conditions. However, in response to oxidative stress including RT, calreticulin is translocated to the cell surface, making this peptide a de novo cell surface epitope after radiation therapy and an ideal prototype of a radiation-induced CAR neoantigen. Exemplary radiation-induced cell surface markers comprise calreticulin, TATA-Box Binding Protein Associated Factor 15 (TAF15), Intercellular adhesion molecule-1 (ICAM-1), E-selectin, P-selectin, Glucose-related protein 78 (GRP78), and combinations thereof.

The antigen-specific extracellular domain can also include a spacer linking the Vh and VL chains of the scFV, which can be the hinge region of IgG1 and is sufficient for most scFv-based constructs. Flexible linkers include glycine-serine linkers and Whitlow linkers.

The intracellular domain transmits the immune cell activation signal. The intracellular domain can increase immune cell cytokine production and facilitate immune cell replication. The intracellular domain reduces CAR T cell exhaustion, increases T cell antitumor activity, and enhances survival of CAR T cells in patients. Exemplary intracellular domains comprise co-stimulatory domains, including those from CD27, CD28, CD137 or 4-1BB, CD154 or CD40L, CD244 or 2B4, CD278 or ICOS, CD134 or OX40, CD3-ζ, and combinations thereof, and signaling domains (also called cytotoxicity domains), including those from CD16, DAP10, DAP12, CD28, ICOS, CD27, OX40, CD40L, CD3-ζ, and combinations thereof. A costimulatory domain is derived from the intracellular signaling domains of costimulatory proteins that enhance cytokine production, proliferation, cytotoxicity, and/or persistence in vivo.

Typically, the antigen-specific extracellular domain is linked to the intracellular domain of the CAR by a transmembrane domain, e.g., derived from a CD4, CD8, CD-8 alpha, CD8-beta, CD3-epsilon, CD3-beta, CD28, 4-1BB, OC40, PD-1, LAG-3, CH2CH3 or NKG2D, IgG, CD3-ζ transmembrane domain, or combinations thereof. The transmembrane domain traverses the cell membrane, anchors the CAR to the T cell surface, and connects the extracellular domain to the intracellular signaling domain, thus impacting expression of the CAR on the T cell surface.

CARs may also further comprise one or more spacers. A spacer or hinge connects (i) the antigen-specific extracellular domain to the transmembrane domain, (ii) the transmembrane domain to a costimulatory domain, (Hi) a costimulatory domain to the intracellular domain, and/or (iv) the transmembrane domain to the intracellular domain. For example, inclusion of a spacer domain (e.g., IgG1, IgG2, IgG4, CD28, CD8) between the antigen-specific extracellular domain and the transmembrane domain may affect flexibility of the antigen-binding domain and thereby CAR function. Transmembrane domains, costimulatory domains, and spacers are known in the art.

In a specific aspect, the CAR comprises a tumor antigen binding domain that targets Ganglioside G2 (GD2), coupled to a transmembrane domain from CD8, CD28, CH2CH3 or NKG2D, coupled to an intracellular domain comprising a costimulatory domain from CD27, CD28, CD137, CD154, CD244, CD278, or a combination thereof, and a cytotoxicity domain from CD3ζ, DAP10, DAP12, CD16, or a combination thereof.

In an aspect, the second expressed CAR construct including a second extracellular domain comprising an antigen recognition domain that specifically binds a radiation-induced cell surface marker can comprise a CAR developed against a single neoantigen, 2,4-dinitrophenyl (DNP). This approach uses antibodies/adaptors labeled with DNP, recognized by the CAR and can be directed towards multiple epitopes on the malignant cells. Thus, in an aspect, the second CAR construct of the genetically modified bicistronic immune cell comprises an anti-2,4-dinitrophenyl (DNP) antibody linked to the second transmembrane domain, and wherein the antigen recognition domain that specifically binds a radiation-induced cell surface marker comprises a DNP-label.

As an alternative to the DNP-based construct, a CAR construct incorporating a binding epitope for a tag such as FITC can be used in combination with tag (e.g., FITC) labelled adaptor/antibody. Thus, in an aspect, the second CAR construct of the genetically modified bicistronic immune cell comprises an anti-tag scFV antibody linked to the second transmembrane domain, and wherein the antigen recognition domain that specifically binds a radiation-induced cell surface marker comprises a tag that binds the anti-tag scFV antibody. In specific aspect, the anti-tag scFV antibody is an anti-FITC scFv and the tag is FITC, the anti-tag scFV antibody is an anti-5B9 scFv and the tag is 5B9, or the anti-tag scFV antibody is an anti-peptide neo-epitope (PNE) scFV and the tag is PNE.

The immune cells are genetically modified to express the CAR constructs. Immune cells can be edited such that an expression construct for the CAR construct is inserted into the genome randomly using a viral vector (retrovirus, lentivirus, AAV, etc.) or inserted into a targeted region in the genome of an immune cell using non-viral approaches (electroporation, mRNA, lipid nanoparticle, etc.) coupled with CRISPR/Cas9, TALEN or Zinc finger nucleases. The CAR can be inserted into an endogenous T cell receptor alpha subunit constant gene (TRAC), or an endogenous T cell receptor beta subunit constant gene (TRBC).

The expression vector for the CAR construct can be inserted into the genome of the unmodified T cells using viral or non-viral methods. The viral vectors include retroviruses (including lentivirus), adenovirus and adeno-associated virus. Production of CAR T cells using viral methods to insert the CAR construct are well-known in the art. Methods of making non-viral CAR T cell products are described in US2020/0000851; WO2021/173925 and WO2023/023635, incorporated herein by reference for their disclosure of making non-viral CAR T cell products.

In an exemplary non-viral method as described in WO2021/173925, a Cas9 RNP and a non-viral double-stranded HDR template including the CAR are introduced into the unmodified T cells to provide genome-edited T cells comprising the CAR.

Genome editing of the T cells can employ a CRISPR system, or Cas9 ribonucleoprotein. CRISPR refers to the Clustered Regularly Interspaced Short Palindromic Repeats type II system used by bacteria and archaea for adaptive defense. This system enables bacteria and archaca to detect and silence foreign nucleic acids, e.g., from viruses or plasmids, in a sequence-specific manner. In type II systems, guide RNA interacts with Cas9 and directs the nuclease activity of Cas9 to target DNA sequences complementary to those present in the guide RNA. Guide RNA base pairs with complementary sequences in target DNA. Cas9 nuclease activity then generates a double-stranded break in the target DNA.

Immune cells are generally modified ex vivo, that is outside of the patient, and then the modified immune cells such as CAR T cells are returned to the patient, such as by intravenous infusion, subcutaneous, intratumoral, intraperitoneal or intracerebral ventricular injection.

### TRT

Radiation therapy (RT) is used for both curative and palliative treatments in over 50% of cancer patients. By inducing potentially lethal DNA damage, RT triggers immunogenic tumor cell death characterized by the translocation of calreticulin to the plasma membrane and the release of ATP and HMGB 1 protein in the extracellular milieu. This promotes migration and activation of immune cells in the tumor microenvironment (TME). At sublethal doses, RT also activates the STING/cGAS pathway in tumor cells and stroma resulting in a type I interferon (IFN) response and upregulation of immune cell adhesion molecules on tumor endothelial cells. Moreover, RT increases the expression of MHC-I on tumor cells, potentially facilitating tumor recognition by a patient's own tumor-specific T cells. However, for poorly immunogenic tumors such as pediatric neuroblastoma, low tumor mutation burden translates to few tumor-associated neoantigens and limited potential for endogenous T cell recognition.

Targeted radionuclide therapy (TRT) is a growing class of cancer therapeutics that selectively deliver RT to malignant cells in vivo using a tumor-selective ligand (small molecule or antibody) labeled with a radionuclide. Following intravenous injection of a TRT agent, it accumulates in tumor and as the radionuclide undergoes decay, RT is delivered to TMEs throughout the body with markedly less toxicity than whole-body RT. By activating the STING/cGAS pathway, low-dose TRT stimulates a pro-inflammatory response that enhances endogenous T cell infiltration and activation in the TME of solid tumors.

Exemplary TRT agents include metaiodobenzylguanidine (MIBG), where the iodine atom in the MIBG is a radioactive iodine isotope; radiolabeled tumor-targeting antibodies; a radiolabeled tumor-targeting small molecule; a radiolabeled tumor-selective metabolite; a radioactive isotope of radium, such as Ra-223; and radioactive phospholipid ether metal chelates having the formula:

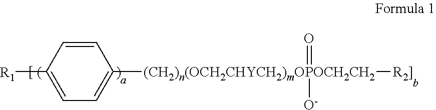

or a salt thereof. R₁ includes (a) a chelating agent that is chelated to a metal atom, wherein the metal atom is an alpha, beta, gamma, or Auger emitting metal isotope with a half-life of greater than 6 hours and less than 30 days or (b) a radioactive halogen isotope; a is 0 or 1; n is an integer from 12 to 30; m is 0 or 1; Y is —H, —OH, —COOH, —COOX, —OCOX, or —OX, wherein X is an alkyl or an arylalkyl; R₂ is —N⁺H₃, —N⁺H₂Z, —N⁺HZ₂, or −N⁺Z₃, wherein each Z is independently an alkyl or an aryl; and b is 1 or 2. In some embodiments, when R₁ is (a) a chelating agent that is chelated to a metal atom, wherein the metal atom is an alpha, beta, gamma, or Auger emitting metal isotope with a half-life of greater than 6 hours and less than 30 days metal isotopes that could be used include Sc-47, Lu-177, Y-90, Ho-166, Re-186, Re-188, Cu-67, Au-199, Rh-105, Ra-223, Ac-225, Pb-212, and Th-227.

In some embodiments, when R₁ is a (b) a radioactive halogen isotope, the radioactive halogen isotope is ¹²³I, ¹²⁴I, ¹²⁵I, ¹³¹I, ²¹¹At, ⁷⁶Br, or ⁷⁷Br. In some embodiments, a is 1 and m is 0. In some embodiments, n is 18. In some embodiments, R₂ is —N⁺ (CH₃)₃. In some such embodiments, a is 1, m is 0, and n is 18. In some such embodiments, the radioactive halogen isotope is ¹²³I, ¹²⁴I, ¹²⁵I, or ¹³¹I (the radiohalogenated phospholipid ether is [¹²³I]-NM404, [¹²⁴I]-NM404, [¹²⁵I]-NM404, [¹³¹I]-NM404, [²¹¹At]-NM404, [⁷⁶Br]-NM404, or [₇₇Br]-NM404).

In an aspect, (i) m is 0, b is 1, n is an integer from 12 to 30, and R₂ is —N⁺Z₃, wherein each Z is independently an alkyl or an aryl; or (ii) m is 1, b is 1, n is an integer from 12 to 30, and R₂ is —N⁺Z₃, wherein each Z is independently an alkyl or an aryl; or (iii) m is 0, b is 1, n is 18, and R₂ is —N⁺Z₃, wherein each Z is independently an alkyl or an aryl; or (iv) m is 1, b is 1, n is 18, and R₂ is —N⁺Z₃, wherein each Z is independently an alkyl or an aryl.

Exemplary chelating agents include 1,4,7,10-tetraazacyclododecane-1,4,7-triacetic acid (DO3A) or one of its derivatives; 1,4,7-triazacyclononane-1,4-diacetic acid (NODA) or one of its derivatives; 1,4,7-triazacyclononane-1,4,7-triacetic acid (NOTA) or one of its derivatives; 1,4,7,10-tetraazacyclododecane-1,4,7,10-tetraacetic acid (DOTA) or one of its derivatives; 1,4,7-triazacyclononane, 1-glutaric acid-4,7-diacetic acid (NODAGA) or one of its derivatives; 1,4,7,10-tetraazacyclodecane, 1-glutaric acid-4,7,10-triacetic acid (DOTAGA) or one of its derivatives; 1,4,8,11-tetraazacyclotetradecane-1,4,8,11-tetraacetic acid (TETA) or one of its derivatives; 1,4,8,11-tetraazabicyclo[6.6.2]hexadecane-4,11-diacetic acid (CB-TE2A) or one of its derivatives; diethylene triamine pentaacetic acid (DTPA), its diester, or one of its derivatives; 2-cyclohexyl diethylene triamine pentaacetic acid (CHX-A″-DTPA) or one of its derivatives; deforoxamine (DFO) or one of its derivatives; 1,2-[[6-carboxypyridin-2-yl]methylamino]ethane (H₂dedpa) or one of its derivatives; and DADA or one of its derivatives, wherein DADA has the structure:

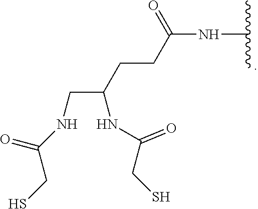 [SMILES: *NC(=O)CCC(CNC(=O)CS)NC(=O)CS]

In some embodiments, a is 1 (aliphatic aryl-alkyl chain). In other embodiments, a is 0 (aliphatic alkyl chain). In some embodiments, m is 1 (acylphospholipid series). In some such embodiments, n is an integer between 12 and 20. In some embodiments, Y is —OCOX, —COOX or —OX. In some embodiments, X is —CH₂CH₃ or —CH₃. In some embodiments, m is 0 (alkylphospholipid series). In some embodiments, b is 1. In some embodiments, n is 18.

In some embodiments, R₂ is —N⁺Z₃. In some such embodiments, each Z is independently —CH₂CH₃ or —CH₃. In some such embodiments, each Z is —CH₃.

In some embodiments, the chelating agent chelated to the metal atom is:

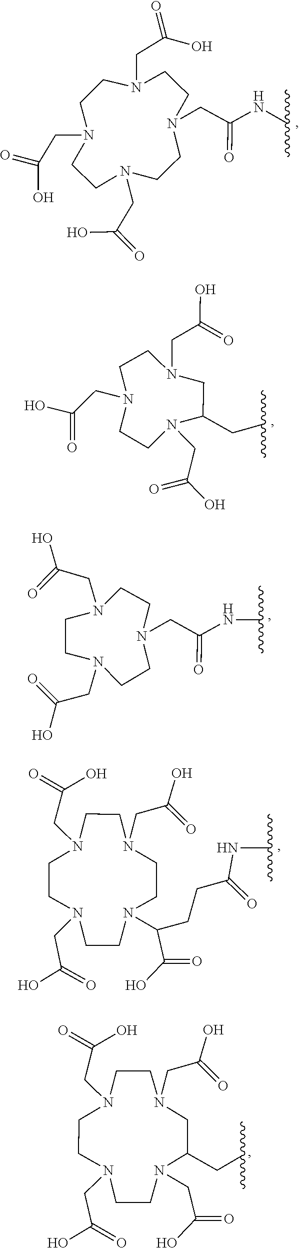

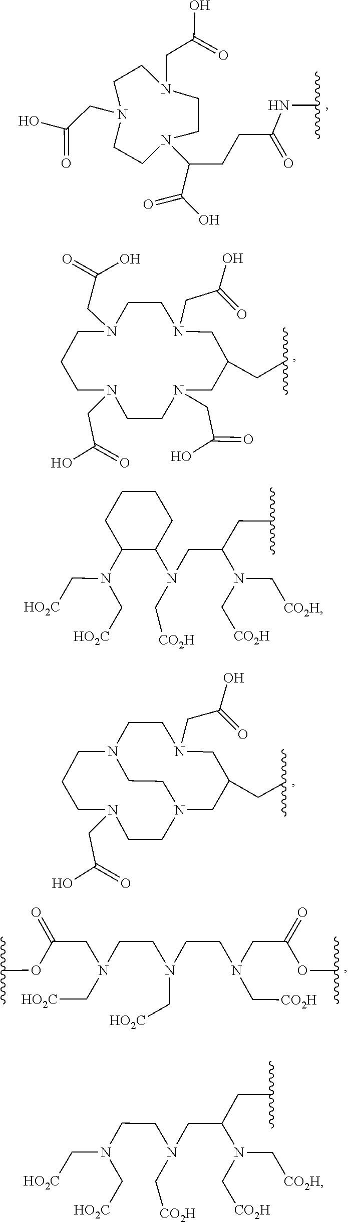

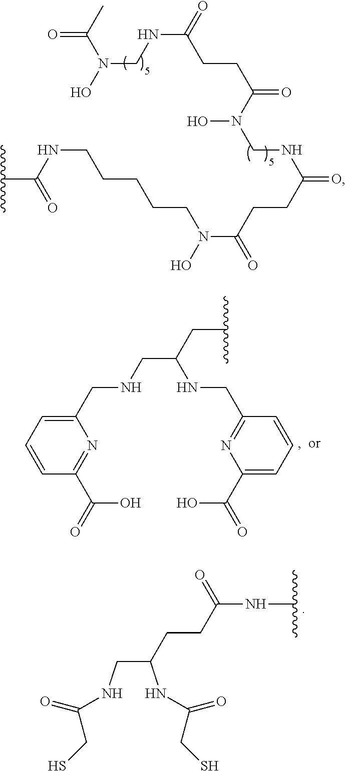

In some embodiments, the chelating agent chelated to the metal atom is:

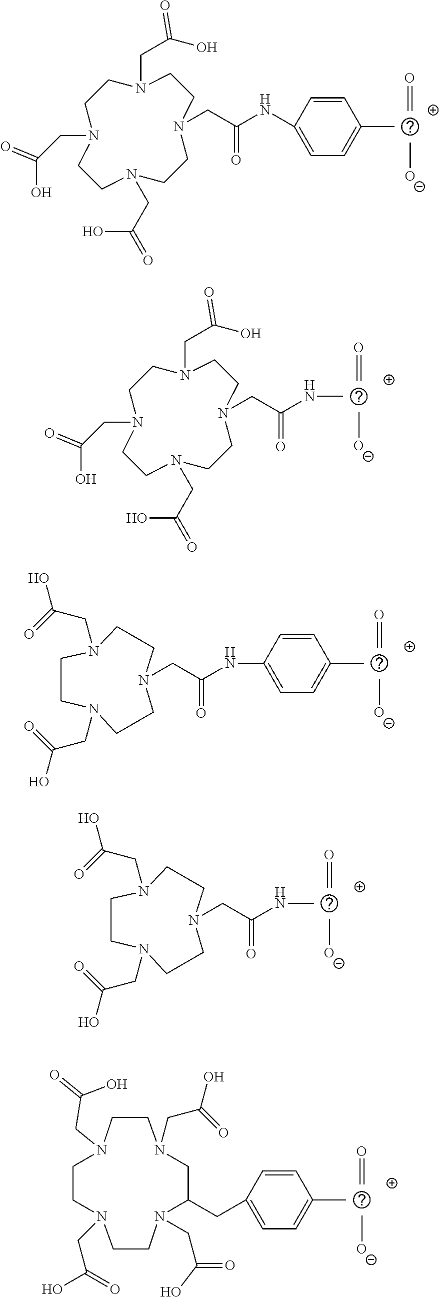

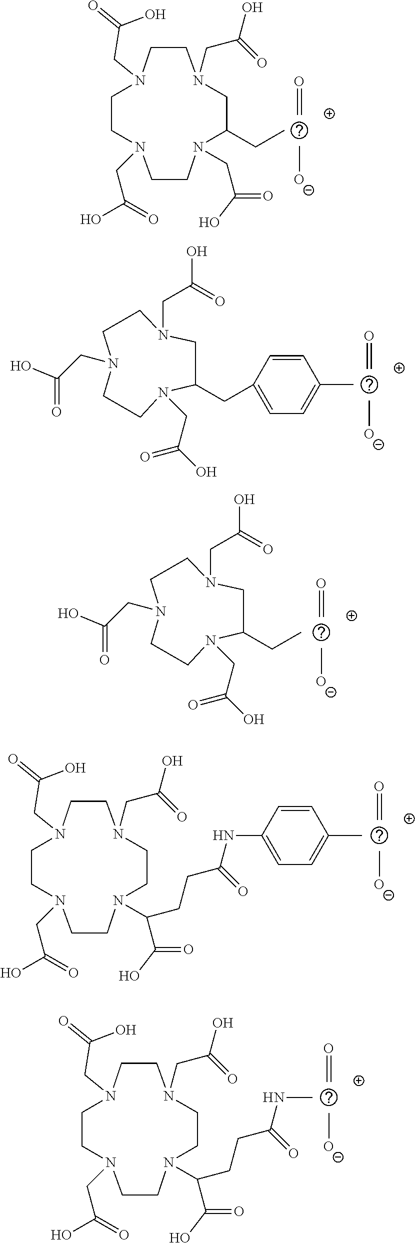

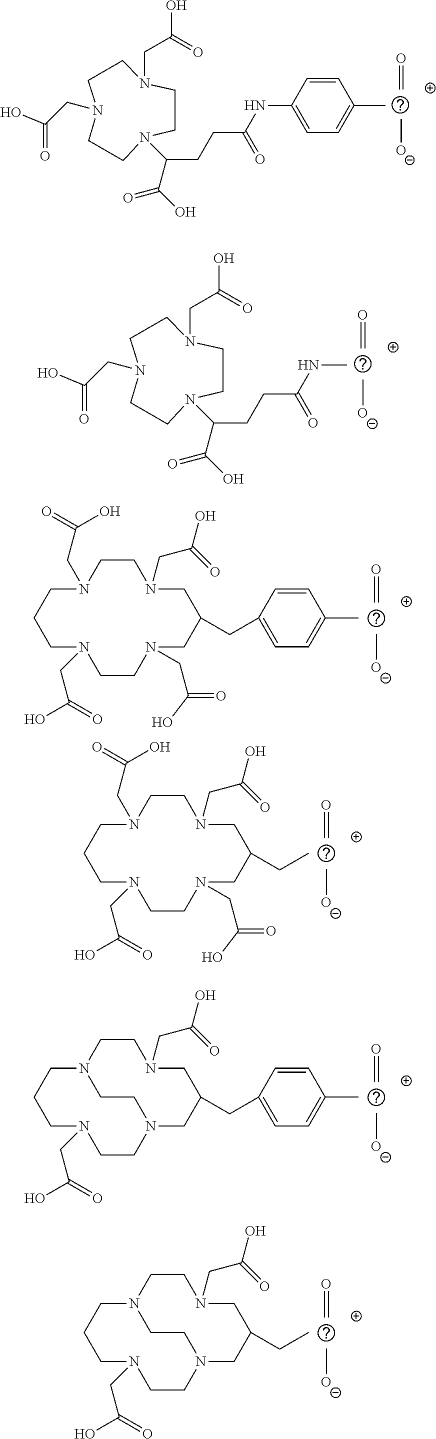

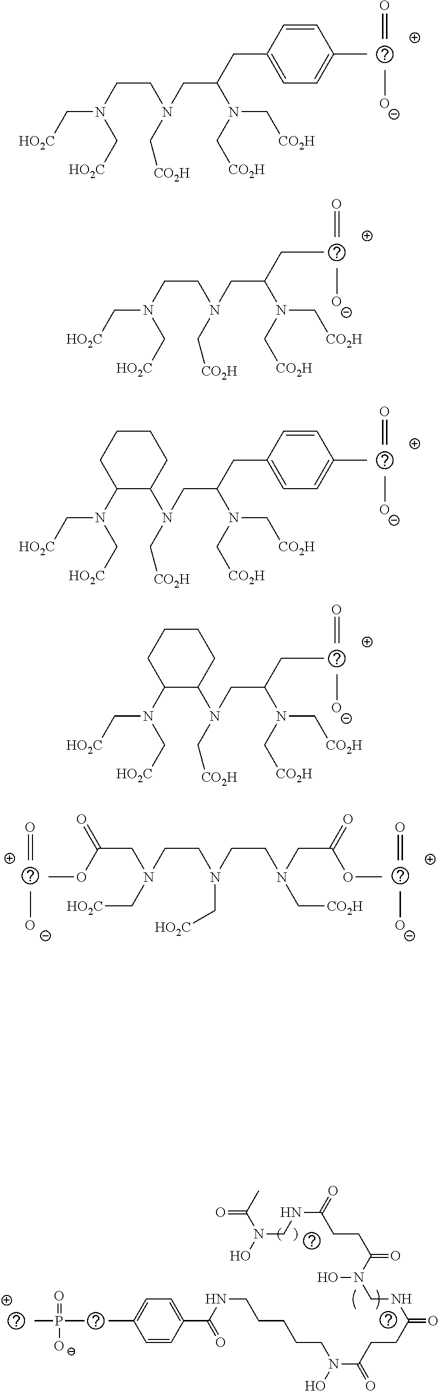

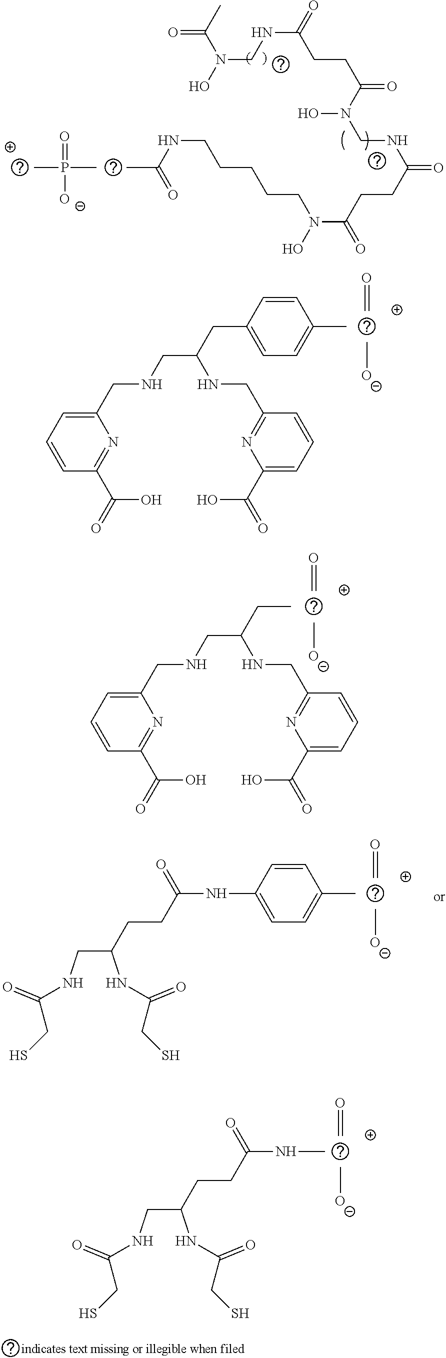

In some embodiments, in the phospholipid ether metal chelate structure, a is 1, b is 1, m is 0, n is 18, and R₂ is —N⁺(CH₃)₃. In some such embodiments, the phospholipid ether metal chelate is NM600 chelated to the metal atom, such as (but not limited to) ⁹⁰Y-NM600. NM600 with ⁶⁴Cu as the metal atom is shown below:

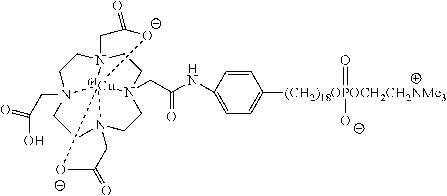

In another aspect, the TRT agent is metaiodobenzylguanidine (MIBG), where the iodine atom in the MIBG is a radioactive iodine isotope.

In another aspect, the TRT agent is a radiolabeled tumor-targeting antibody. Exemplary radiolabeled tumor-targeting antibodies include ¹⁷⁷Lu-girentuximab, rosopatamab radiolabeled with yttrium-90 and lutetium-177, ¹³¹I-labetuzumab, Panitumumab conjugated to α-emitter, ²¹²Pb, ⁹⁰Y labeled anti-MUC1 antibody, nti-TAG-72 intact antibodies radiolabeled with iodine-131, yttrium-90, and lutetium-177, ⁹⁰Y-clivatuzumab tetraxetan, ³¹I-labeled mAb 81C6, and the like.

In another aspect, the TRT agent is a radioactive isotope of radium such as radium-223 dichloride.

Radionuclides are radioactive atoms and historically for TRT patients have been prescribed a given activity (unit curie, Ci), which is the rate of disintegration or radioactive decay. For research and mechanism-driven therapeutic approaches, however, it the absorbed dose (unit gray: Gy) of RT that will be administered from a given activity is determined. This is because absorbed dose, which is defined as the energy absorbed per unit mass of tissue, mediates the biological effects of radionuclides.

Without being held to theory, the radiation doses (Gy) described herein for tumors implanted in mice should translate to humans because the tumors implanted were derived from human cancer cell lines. As is known in the art, the optimal radiation dose can vary from one tumor to the other, however, in general, a low dose of a TRT agent is a dose that is less than the typical dose used for TRT monotherapy, that is, the dose of TRT expected to kill tumor cells.

In an aspect, the dose of a TRT agent provides a 0.25 Gy to 20 Gy either in a single fraction or in fractions delivering 1.8 to 12 Gy per fraction, to a cumulative dose of up to 80 Gy. The disclosed doses are suitable for a human patient, for example, including a child or an adult.

### Dosing Regimens

In an aspect, a method of treating a cancer in a subject comprises administering to the subject a dose of a targeted radionuclide therapy (TRT) agent, and administering to the subject a genetically modified bispecific immune cell therapy as described herein.

The genetically modified bispecific immune cell therapy can be administered prior to, concurrent with, or after the administration of the TRT agent.

In an aspect, the TRT therapy is administered prior to the genetically modified bispecific immune cell therapy. The time period for waiting between therapies is based on the half-life of the TRT agent and the starting radiation dose. Because the TRT is expected to decrease the viability of the genetically modified bispecific immune cells, the waiting period can be an important part of the method. Exemplary waiting periods include 1 to 60 days, 1 to 30 days, 2 to 30 days, 2 to 20 days, 2 to 12 days, 3 to 10 days, 3 to 9 days or 3 to 6 days.

In an aspect, the method further comprises administering external beam sources of radiation therapy such as x-rays, protons, electrons, neutrons, carbon ions, and combinations thereof.

In an aspect, the subject is a mammalian subject, specifically a human or canine subject.

In an aspect, the compositions and methods described herein are particularly useful to treat cancers such as breast cancer, neuroblastoma, melanoma, sarcoma, neuroendocrine cancer, colorectal cancer, lung cancer, head and neck cancer, prostate cancer, pancreatic cancer, ovarian cancer, glioblastoma, lymphoma, diffuse midline glioma, or a combination thereof. In an aspect, the cancer is a poorly immunogenic solid tumor.

The disclosure is inclusive of the compounds described herein (including intermediates) in any of their pharmaceutically acceptable forms, including isomers (e.g., diastereomers and enantiomers), tautomers, salts, solvates, polymorphs, prodrugs, and the like. It should be understood that the term “compound” includes any or all of such forms, whether explicitly stated or not (although at times, “salts” may be explicitly stated).

“Pharmaceutically acceptable” as used herein means that the compound or composition or carrier is suitable for administration to a subject to achieve the treatments described herein, without unduly deleterious side effects in light of the necessity of the treatment.

The term “effective amount,” as used herein, refers to the amount of the compounds or dosages that will elicit the biological or medical response of a subject, tissue or cell that is being sought by the researcher, veterinarian, medical doctor or other clinician.

The term, “pharmaceutically-acceptable carrier” includes any and all dry powder, solvents, dispersion media, coatings, antibacterial and antifungal agents, isotonic agents, absorption delaying agents, and the like. Pharmaceutically-acceptable carriers are materials, useful for the purpose of administering the compounds in the method of the present invention, which are preferably non-toxic, and may be solid, liquid, or gaseous materials, which are otherwise inert and pharmaceutically acceptable, and are compatible with the compounds described herein. Examples of such carriers include, without limitation, various lactose, mannitol, oils such as corn oil, buffers such as PBS, saline, polyethylene glycol, glycerin, polypropylene glycol, dimethylsulfoxide, an amide such as dimethylacetamide, a protein such as albumin, and a detergent such as Tween® 80, mono- and oligopolysaccharides such as glucose, lactose, cyclodextrins and starch.

The term “administering” or “administration,” as used herein, refers to providing the compound or pharmaceutical composition of the invention to a subject suffering from or at risk of the diseases or conditions to be treated or prevented.

A route of administration in pharmacology is the path by which a drug is taken into the body. Routes of administration may be generally classified by the location at which the substance is applied. Common examples may include oral and intravenous administration. Routes can also be classified based on where the target of action is. Action may be topical (local), enteral (system-wide effect, but delivered through the gastrointestinal tract), or parenteral (systemic action, but delivered by routes other than the GI tract), via lung by inhalation. One form of local administration is intratumoral (IT), whereby an agent is injected directly into, or adjacent to, a known tumor site.

A topical administration emphasizes local effect, and substance is applied directly where its action is desired. Sometimes, however, the term topical may be defined as applied to a localized area of the body or to the surface of a body part, without necessarily involving target effect of the substance, making the classification rather a variant of the classification based on application location. In an enteral administration, the desired effect is systemic (non-local), substance is given via the digestive tract. In a parenteral administration, the desired effect is systemic, and substance is given by routes other than the digestive tract.

Examples of parenteral administrations may include intravenous (into a vein), e.g. many drugs, total parenteral nutrition intra-arterial (into an artery), e.g., vasodilator drugs in the treatment of vasospasm and thrombolytic drugs for treatment of embolism, intraosseous infusion (into the bone marrow), intra-muscular, intracerebral (into the brain parenchyma), intracerebroventricular (into cerebral ventricular system), intrathecal (an injection into the spinal canal), and subcutaneous (under the skin). Among them, intraosseous infusion is, in effect, an indirect intravenous access because the bone marrow drains directly into the venous system. Intraosseous infusion may be occasionally used for drugs and fluids in emergency medicine and pediatrics when intravenous access is difficult.

The invention is further illustrated by the following non-limiting examples.

### EXAMPLES

### Materials and Methods

Cell lines: Tumor cell lines including but not limited to CHLA-20 cells (human neuroblastoma cells) and M21 (human melanoma cells) were grown in Dulbecco's Modified Eagle Medium (DMEM) with high glucose or Roswell Park Institute Medium (RPMI) supplemented with 10% fetal bovine serum and 1% penicillin-streptomycin and maintained at 37° C. in 5% CO₂. Cell line authentication was done using genomic short tandem repeat analysis (Idexx BioAnalytics) and by cell morphology per ATCC guidelines. Mycoplasma testing was performed on a regular basis to rule out contamination using the Mycoplasma Detection Kit MycoStrip™.

CAR T/NK/macrophages cells: All retroviral based DNP or bicistronic CAR T/NK/macrophages were manufactured from primary immune cells obtained from healthy human donor and cultured in ImmunoCult™-XF T cell Expansion Medium supplemented with 500 U/mL of IL-2 (Peprotech) and maintained 37° C. in 5% CO₂.

In vitro dosimetry: All in vitro dosimetry studies were performed in a 6-well cell culture plate. Serial dilutions of various activity of free ¹⁷⁷Lu or ²²⁵Ac were made in cell culture medium and added to each well. For ¹⁷⁷Lu, thermoluminescent dosimeters (TLDs) were placed under each well. The TLDs were harvested after 1 half-life and analyzed by the University of Wisconsin-Madison Radiation Calibration Laboratory (Calibration Cert #1664.01). A standard curve was obtained and used to determine the activity of ¹⁷⁷Lu needed to deliver a given radiation dose, as previously reported in the art. To confirm the validity of this method, the mean absorbed dose to cells was calculated using the Geant4 Monte Carlo toolkit and using an extension of RAPID. A model of a flat bottom 6-well plate has been developed in Geant4 using manufacturing specifications where the diameter of each well is 36 mm, and the height of each well is 10.7 mm. The cell volume can be defined as a thin water-equivalent layer at the bottom of the well.

Tumor cell irradiation: The tumor cells were irradiated with various doses of radiation delivered over 3 days by ¹⁷⁷Lu or ²²⁵Ac diluted in 3 mL of cell culture medium including DMEM. Doses of radiation delivered by ¹⁷⁷Lu were 1, 2 and 6 Gy whereas with ²²⁵Ac, 1 and 2 Gy were delivered.

In vitro CAR T/NK/macrophage cells and tumor cells co-culture: After the tumor cells were irradiated, they were harvested and washed 3 times with PBS and co-cultured with CAR T/NK/macrophage cells with an effector to target (E:T) ratio of 10:1 for 24 hrs, in presence of DNP-labelled antibody or unlabeled antibody.

Cytotoxic activity of CAR T/NK/macrophage: The cytotoxic activity of the CAR T/NK/macrophage against irradiated tumor cells expressing the radiation-induced antigen was measured by flow cytometry. For flow cytometry analysis, after the co-culture, all cells were harvested, washed with PBS, and resuspended into single cell solution in PBS. Fc blocking (Biolegend, 422302) and Live/Dead staining with Ghost Dye™ Red 780 (Tonbo Biosciences, 13-0865-T100) were performed for 10 minutes at 4° C. The fluorophore-conjugated anti-CD45-APC (Biolegend, 304012) was incubated for 20 minutes at 4° C. and washed with 2% FBS in PBS. The analysis of the sample was performed using the Attune™ NxT Flow Cytometer (ThermoFisher) and the collected data was analyzed using FlowJo software.

Statistical analysis: All statistical analysis were performed in Prism 9 (GraphPad Software). Two-way ANOVA with Tukey's multiple comparisons test was used for comparison between multiple groups.

### Example 1: Develop CAR T Cells Targeting ⁹⁰Y-NM600 TRT-Induced Calreticulin Neoantigen on the Cell Surface of GD2 Expressing Malignant Cells

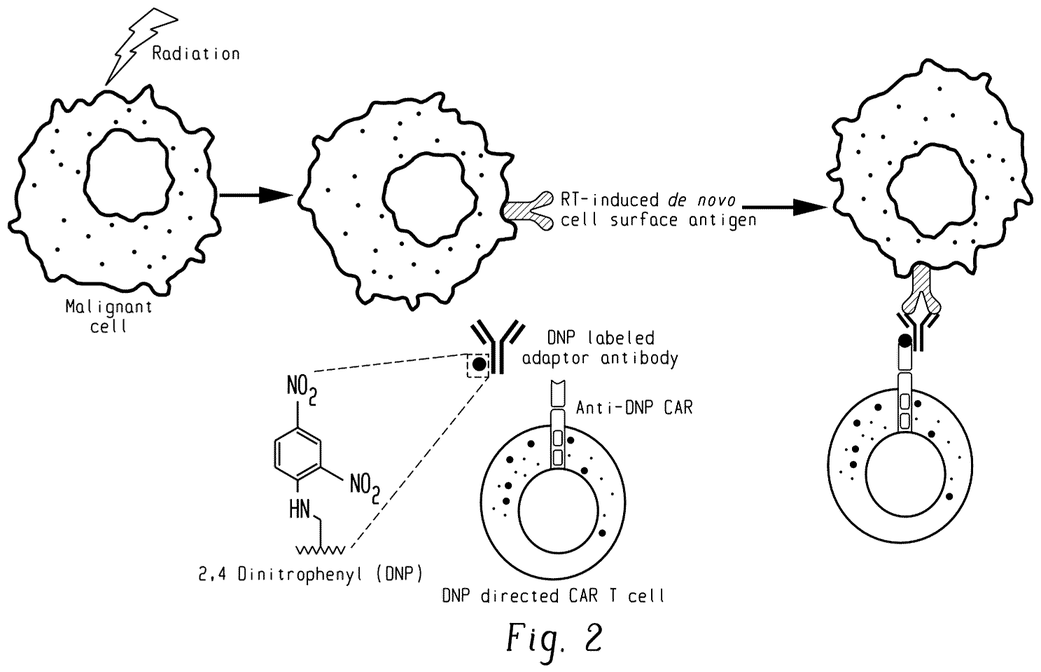

A CAR has been developed against a single neoantigen, 2,4-dinitrophenyl (DNP). This approach uses antibodies/adaptors labelled with DNP, recognized by the CAR, and can be directed towards multiple epitopes on the malignant cells (FIG. 2). In this first set of experiments, calreticulin will be targeted using this approach to demonstrate its feasibility, thus enabling the targeting of other neoantigens using the DNP directed CAR.

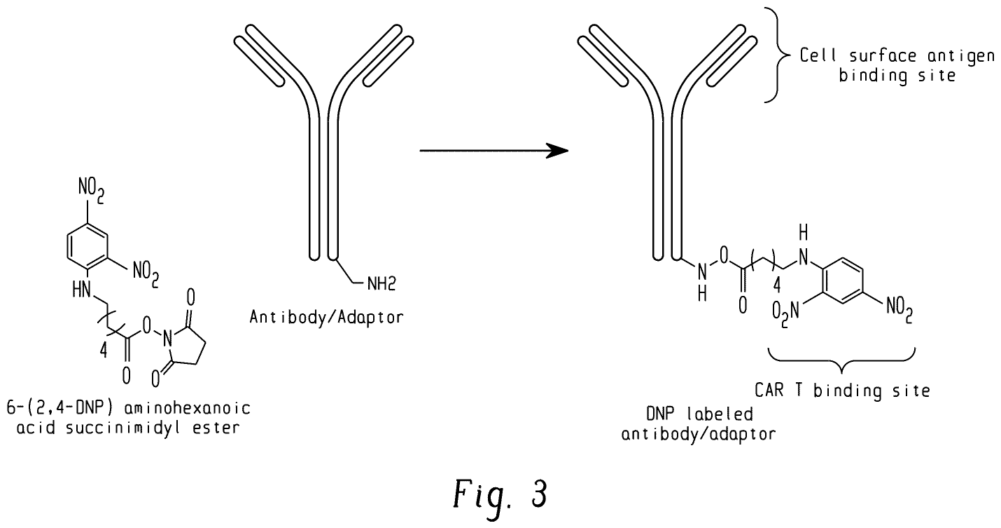

More specifically, a DNP-labelled anti-calreticulin antibody/adaptor will enable the targeting of cells expressing calreticulin on the plasma membrane by CAR T cells designed to recognize DNP. Commercially available anti-calreticulin antibody will be conjugated with DNP as previously reported to yield a DNP-labelled anti-calreticulin antibody (FIG. 3). The conjugation of DNP to the anti-calreticulin antibody will be verified by Western blot analysis using an anti-DNP secondary antibody.

CHLA-20 neuroblastoma and M21 melanoma cells expressing GD2 will be exposed to various dose of ⁹⁰Y-NM600 TRT, ranging from 1 to 5 Gy. Using flow cytometry, the translocation of calreticulin on the cell surface will be monitored at various time points (12, 24, 48, 72, 96, 120 hours) after the ⁹⁰Y-NM600 radiation exposure. The comparison of the cell surface calreticulin between non-irradiated and irradiated malignant cells with the various radiation doses will enable us to identify the optimal ⁹⁰Y-NM600 dose needed and the adequate post-radiation incubation time required for maximal cell surface translocation of calreticulin. This time-dependence study will enable identification of when the calreticulin translocated to the cell surface is degraded, thus allowing determination of the time point at which ⁹⁰Y-NM600 TRT can be repeated in order to achieve a cyclical translocation of calreticulin and consequently a cyclical activation of CAR T cells targeting a radiation-induced epitope.

To evaluate the efficacy of the design CAR T cells and the DNP-labelled anti-calreticulin antibody/adaptor, a cell line with constitutive expression of cell surface calreticulin will be generated. HEK293 cells will be transfected with a plasmid encoding for GFP-Calreticulin, a N-terminal GFP fusion with calreticulin (amino acids 18-417). In this construct, the ER targeting sequence of calreticulin (amino acids 1-17) is absent, resulting in high level of cytosolic and cell surface calreticulin. The cell surface expression of calreticulin will be confirmed by immunofluorescence detection of GFP and by flow cytometry with fluorophore labelled secondary antibody directed against calreticulin. The activation of CAR T cells by the DNP-labelled anti-calreticulin antibody will be evaluated in HEK293 cells expressing cell surface calreticulin (CS-CRT⁺) compared to non-transfected (CS-CRT⁻) HEK293 cells by measuring cytokines production using Luminex® cytokine profile assays. After an incubation of the CS-CRT⁺ HEK293 or CS-CRT⁻ HEK293 cells with the DNP-labelled anti-calreticulin antibody or an isotype control, the CS-CRT⁺ and CS-CRT⁻ HEK293 cells will be co-cultured at a 1:1 ratio with DNP-directed CAR T cells. After incubation, the supernatant will be collected and the expression of diverse cytokines will be determined by Luminex® assay. The cytotoxic activity of the DNP-directed CAR T cells mediated by the DNP-labelled anti-calreticulin antibody/adaptor will also be evaluated using Annexin V/Caspase-9 staining by the Incucyte® live-cell analysis system. DNP-directed CAR T cells will be incubated with CS-CRT⁺ HEK293 or CS-CRT⁻ HEK293 cells at various ratio (T cells: HEK293; 1:64; 1:32; 1:16; 1:8; 1:4; 1:2; 1:1) and fixed concentration (10 nM) of the DNP-labelled anti-calreticulin antibody/adaptor. The percent cytotoxicity will subsequently be calculated.

### Example 2: Screening of Radiation-Induced Neoantigens

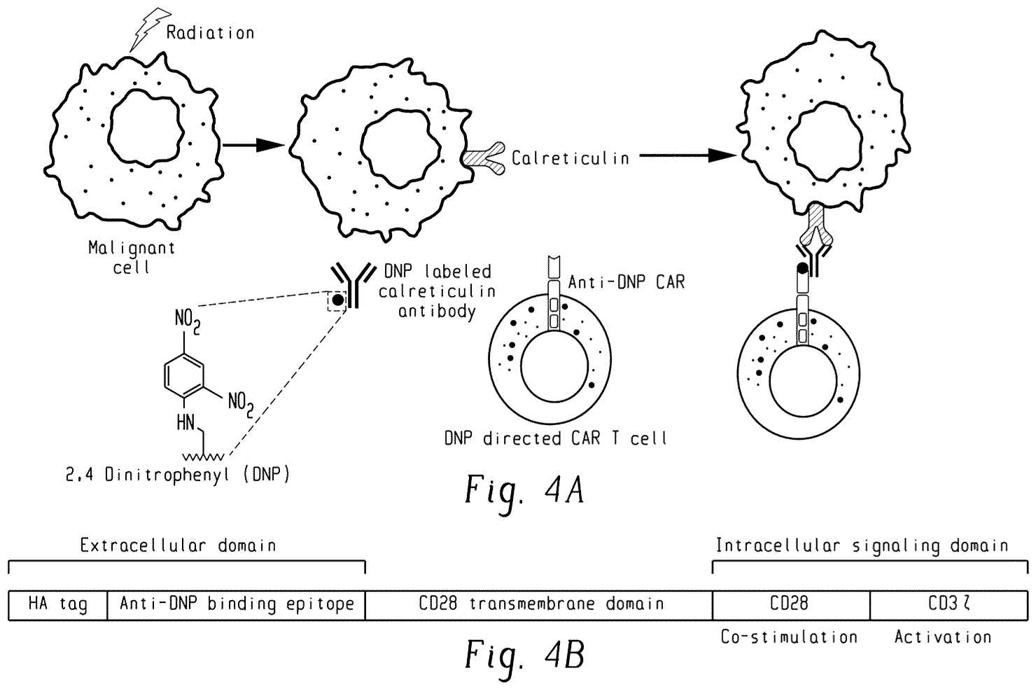

To enable the screening of various radiation-induced neoantigens, the CAR T cell engineered to bind DNP will be used. DNP-labelled anti-calreticulin antibody will be used to redirect CAR T cells toward malignant cells expressing calreticulin as a cell surface neoantigen (FIG. 4A). Using a previously described anti-DNP CAR construct, CAR T cells will be obtained after the expression of anti-DNP CAR on T cells isolated from human donors. This construct utilizes an extracellular anti-DNP binding domain, a CD28 transmembrane domain and a second-generation CAR intracellular signaling domain comprised of an activation domain (CD35) and a CD28 co-stimulatory domain (FIG. 4B). This construct will be used to generate retroviral particles. T cells will be isolated from healthy human donor using a negative selection and after ex vivo expansion, will be transduced with the generated lentiviral particles to yield the anti-DNP CAR T cells. The expression of the CAR will be confirmed by flow cytometry.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A genetically modified bispecific immune cell, comprising
a genetically modified bicistronic immune cell comprising
a first expressed chimeric antigen receptor (CAR) construct comprising a first extracellular domain linked to a first intracellular domain through a first transmembrane domain, wherein the first extracellular domain comprises an antigen recognition domain that specifically binds a tumor-specific antigen, and
a second expressed CAR construct comprising a second extracellular domain linked directly or indirectly to a second intracellular domain through a second transmembrane domain, wherein the second extracellular domain comprises an antigen recognition domain that specifically binds a radiation-induced cell surface marker,
or
a genetically modified bivalent immune cell comprising a bivalent expressed CAR construct comprising a bivalent extracellular domain which comprises a first antigen recognition domain that specifically binds a tumor-specific antigen and a second antigen recognition domain that specifically binds a radiation-induced cell surface marker, linked to a single intracellular domain through a single transmembrane domain.

2. The genetically modified bispecific immune cell of claim 1, wherein the immune cell is a T-cell, a Natural Killer (NK) cell, an innate lymphoid cell, a Cytokine Induced Killer (CIK) cell, a hematopoietic progenitor cell, a peripheral blood (PB) derived immune cell, a bone marrow derived immune cell, a macrophage, or an umbilical cord blood (UCB) derived immune cell.

3. The genetically modified bispecific immune cell of claim 2, wherein the immune cells are modified autologous cells isolated from a patient in need of cancer treatment, or modified cells from an allogeneic healthy donor.

4. The genetically modified bispecific immune cell of claim 1, wherein the second CAR construct of the genetically modified bicistronic immune cell comprises an anti-2,4-dinitrophenyl (DNP) antibody linked to the second transmembrane domain, and wherein the antigen recognition domain that specifically binds a radiation-induced cell surface marker comprises a DNP-label.

5. The genetically modified bispecific immune cell of claim 1, wherein the second CAR construct of the genetically modified bicistronic immune cell comprises an anti-tag scFV antibody linked to the second transmembrane domain, and wherein the antigen recognition domain that specifically binds a radiation-induced cell surface marker comprises a tag that binds the anti-tag scFV antibody.

6. The genetically modified bispecific immune cell of claim 5, wherein the anti-tag scFV antibody is an anti-FITC scFv and the tag is FITC, the anti-tag scFV antibody is an anti-5B9 scFv and the tag is 5B9, or the anti-tag scFV antibody is an anti-peptide neo-epitope (PNE) scFV and the tag is PNE.

7. The genetically modified bispecific immune cell of claim 1, wherein the tumor-specific antigen comprises carbonic anhydrase IX (CAIX), carcinoembryonic antigen (CEA), CD5, CD8, CD7, CD10, CD19, CD20, CD22, CD30, CD33, CLL1, CD34, CD38, CD41, CD44, CD49f, CD56, CD74, CD133, CD138, CD123, CD44V6, mesothelin, Claudin-18, B7 homolog 3 protein (B7-H3), fibroblast activation protein (FAP), cancer antigen 19 (CA19), an antigen of a cytomegalovirus (CMV) infected cell, epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), adult AChR subunits, folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-vIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A, ERBB, or a combination thereof.

8. The genetically modified bispecific immune cell of claim 7, wherein the tumor-specific antigen comprises a tumor antigen selected from GD2, HER2, EGFR, mesothelin, Claudin-18.2, PSMA, B7-H3, IL-13Rα2, FAP, CA19, CD19, CD5, MUC1, or a combination thereof.

9. The genetically modified bispecific immune cell of claim 1, wherein the radiation-induced cell surface marker comprises calreticulin, TATA-Box Binding Protein Associated Factor 15 (TAF15), Intercellular adhesion molecule-1 (ICAM-1), E-selectin, P-selectin, Glucose-related protein 78 (GRP78), or a combination thereof.

10. The genetically modified bispecific immune cell of claim 1, wherein the first and second transmembrane domains independently comprise CD4, CD8, CD-8 alpha, CD8-beta, CD3-epsilon, CD3-beta, CD28, 4-1BB, OC40, PD-1, LAG-3, CH2CH3 or NKG2D, IgG, CD3-ζ, or a combination thereof, or wherein the single transmembrane domain comprises CD4, CD8, CD-8 alpha, CD8-beta, CD3-epsilon, CD3-beta, CD28, 4-1BB, OC40, PD-1, LAG-3, CH2CH3 or NKG2D, IgG, CD3-ζ, or a combination thereof.

11. The genetically modified bispecific immune cell of claim 1, wherein the first and second intracellular domains independently comprise a costimulatory domain selected from CD27, CD28, CD137, CD154, CD244, CD278, or a combination thereof, and a cytotoxicity domain selected from CD3ζ, DAP10, DAP12, CD16, or a combination thereof; or wherein the single intracellular domain comprises a costimulatory domain selected from CD27, CD28, CD137, CD154, CD244, CD278, or a combination thereof, and a cytotoxicity domain selected from CD35, DAP10, DAP12, CD16, or a combination thereof.

12. A method of treating a solid tumor in a subject, comprising
administering to the subject a dose of a targeted radionuclide therapy (TRT) agent, and
administering to the subject the genetically modified bispecific immune cell of claim 1.

13. The method of claim 12, comprising waiting a period of 1 to 60 days after administering the TRT agent, and after waiting, administering the genetically modified bispecific immune cell.

14. The method of claim 13, wherein the waiting period is 1 to 30 days.

15. The method of claim 12, wherein the dose of the TRT agent is a 0.25-20 Gy radiation dose.

16. The method of claim 12, wherein the dose of the TRT agent is delivered in fractions of 1.8 to 12 Gy per fraction, to a cumulative dose of up to 80 Gy.

17. The method of claim 12, further comprising administering an external source of radiation therapy selected from x-rays, protons, electrons, neutrons, carbon ions, and combinations thereof.

18. The method of claim 12, wherein the cancer is a poorly immunogenic solid tumor.

19. The method of claim 12, wherein the cancer is breast cancer, neuroblastoma, melanoma, sarcoma, neuroendocrine cancer, colorectal cancer, lung cancer, head and neck cancer, prostate cancer, pancreatic cancer, ovarian cancer, glioblastoma, lymphoma, diffuse midline glioma, or a combination thereof.

20. The method of claim 12, wherein the TRT agent is metaiodobenzylguanidine (MIBG), where the iodine atom in the MIBG is a radioactive iodine isotope; a radiolabeled tumor-targeting antibody; a radiolabeled tumor-targeting small molecule; a radiolabeled tumor-selective metabolite; a radioactive isotope of radium; or a radioactive phospholipid ether metal chelate.

21. The method of claim 20, wherein the radioactive phospholipid ether metal chelate has the formula 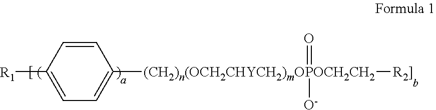
wherein
R₁ is s (a) a chelating agent that is chelated to a metal atom, wherein the metal atom is an alpha, beta or Auger emitting metal isotope with a half-life of greater than 6 hours and less than 30 days or (b) a radioactive halogen isotope,
a is 0 or 1;
n is an integer from 12 to 30;
m is 0 or 1;
Y is —H, —OH, —COOH, —COOX, —OCOX, or —OX, wherein X is an alkyl or an arylalkyl;
R₂ is —N⁺H₃, —N⁺H₂Z, —N⁺HZ₂, or —N⁺Z₃, wherein each Z is independently an alkyl or an aryl, and
b is 1 or 2.

22. The method of claim 20, wherein the radioactive phospholipid ether metal chelate is NM600 chelated to the metal atom.

23. The method of claim 22, wherein the radioactive phospholipid ether metal chelate is ⁹⁰Y-NM600.
